(19) United States
(12) Patent Application Publication
(10) Pub. No.: US 2002/0188263 A1
(43) Pub. Date: Dec. 12, 2002

(54) Body fluid collection device

(76) Inventors: Tuan Le Bui, Round Lake Beach, IL (US); Julieta Ildefonso, Elmwood Park, IL (US)

Correspondence Address: OLSON & HIERL, LTD., 36th Floor, 20 North Wacker Drive, Chicago, IL, 60606, US

(21) Appl. No.: 10/135,654

(22) Filed: Apr. 30, 2002

Related U.S. Application Data
(60) Provisional application No. 60/289,380, filed on May 8, 2001.

(51) Int. Cl. A61F 13/20; A61F 15/00

(52) U.S. Cl. 604/358; 602/56

(57) ABSTRACT

A composite collection device suitable for the collection of body fluid samples is disclosed, The device is particularly suitable for the collection of whole blood for the determination of analytes therein. The composite body fluid collection device comprises a liquid absorbent web and a liquid distributing overlayer contiguous with said absorbent web, the liquid distributing overlayer being liquid permeable and providing flux through the overlayer of no more than about 4 microliters/square millimeter/second.

### CROSS-REFERENCE TO RELATED APPLICATIONS

This application claims priority to provisional application for patent Serial No. 60/289,380 filed on May 8, 2001.

### TECHNICAL FIELD OF THE INVENTION

This invention relates to the field of sampling of body fluids and, more particularly, to body fluid sample collection devices.

### BACKGROUND OF THE INVENTION

Collecting a blood specimen employing blood collection paper for the purpose of determining the presence or quantity of a blood analyte is commonly practiced in medicine. Example analytes include prostate specific antigen, phenylalanine, bone alkaline phosphatase, and hemoglobin Alc for determining, screening, or monitoring prostate cancer, phenylketonuria, osteoporosis, and diabetes, respectively.

Blood collection paper is designed to absorb blood and to contain the absorbed blood. In most cases, the blood is allowed to dry in the paper. The paper containing the blood (dried or not dried) is sent to an analytical laboratory, often by mail, for testing. In most, if not all, cases analytes are worked up in the laboratory by punching coupons from the paper.

Blood collection paper is relatively inexpensive compared with other blood sample collection products. The collection of blood samples in blood collection paper has traditionally been done by healthcare professionals (professional collection). However an increasing number of products that employ blood collection paper are becoming available where a layperson collects his or her own blood sample (self collection) or assists in the collection of another person's blood sample (assisted collection).

One of the most widely used blood collection papers is S&S® 903™ blood collection paper or membrane made of cotton fibers. For example, a “Neonatal Screening Card” employed by the State of Illinois for professional blood sample collection is comprised of S&S® 903™ blood collection paper (Schleicher & Schuell, Keene, N.H.), a protective flap, and an authorization record form. Another example is the “Biosafe Blood Collection Card” (Biosafe Laboratories, Inc., Chicago, Ill.), which is comprised of S&S® 903™ blood collection paper and can be used for self, assisted, and professional collection of blood samples.

A major application of blood collection paper is for absorbing capillary blood specimens. A less common application is for absorbing venous or arterial blood specimens and this application is usually employed when the venous or arterial specimen is to be used for research purposes or when the specimen is to be mailed, stored, or stabilized.

For capillary specimens the skin is typically punctured with a lancet, and the resulting blood is usually applied to the paper by contacting a formed hanging, sessile, or standing drop of blood with the paper (See, for example, “Tietz Textbook of Clinical Chemistry-2nd Ed”, Carl A. Burtis & Edward R. Ashwood editors, 1994, pages 64-65, ISBN 0-7216-4472-4(“Tietz”) the relevant of disclosure of which is incorporated herein by reference.

For capillary specimens, blood collection paper can be filter paper, usually marked with one or more sample receiving regions (typically circular regions). The user is ordinarily instructed to completely fill the area of each marked sample receiving region with blood. The marked sample receiving region also serves as a psychological target for the user, as well as to indicate to the user when sufficient blood has been applied to the paper.

Despite the attempts of some medical organizations to adopt universal procedures for the collection of capillary specimens in blood collection paper, it is clear that different products employ different procedures. For example, in some procedures the user is instructed to make only a single application of blood per sample receiving region. In other procedures, the user is instructed to apply, if necessary, more than one drop of blood per sample receiving region but to apply each drop separately onto open areas of each sample receiving region and not to layer the drops of blood on top of one another. The instructions of still other procedures do not clearly indicate the number of drops of blood to be applied but warn the users not to over-saturate the sample receiving region.

Sometimes the user is specifically instructed to make sure that the blood has completely or totally soaked through the paper while in other procedures such instruction is not given.

In some procedures, the user is specifically warned to avoid handling the paper due to the negative effects of skin oil contamination while in other procedures no such warning is given.

Some procedures instruct the user to place the paper on a clean dry surface while in others the paper is part of a kit that provides a bottom surface.

Tietz is a widely accepted and popular textbook of clinical laboratory chemistry. Tietz teaches that only a single formed drop of blood be applied to a sample receiving region, that the drop completely occupy the region, that the drop completely soak the paper, and that milking or squeezing the puncture wound should be avoided. These instructions are extremely demanding of users, even experienced users, and viewed by many as unrealistic because it forces users to guess or use their best judgment to determine when the blood drop has formed sufficient volume.

Additional issues that complicate capillary blood collection include the variation in bleed rate and volume among subjects, blood clotting, puncture wound size variation, and interferences due to subject and user anxiety. Thus, users often fail to comply with some or all of the aspects of the collection instructions (noncompliance). For example, when users encounter poor bleeders, or when the user is a poor bleeder (in self-collection situations), the user will often spot the application surface of the same sample receiving region multiple times giving the appearance that sufficient blood was present in a single application. When the lab detects noncompliance, the lab can reject the specimen. This process, of course, is costly, time-consuming, and may delay treatment, but more damaging situations can occur when the lab does not detect noncompliance because this may lead to a false positive or false negative result, which results can be harmful to the health welfare of the person.

Blood collection paper is also used for collection of venous or arterial blood specimens. First the blood is withdrawn, usually by venipuncture or arterial puncture, into a container such as an evacuated blood tube. Then, a sample of the blood is transferred to the paper usually with an implement such as a pipette. In some cases a measured volume of blood is transferred from the blood tube to the paper using a volumetric pipette while in other cases the volume of blood that is transferred is not measured. Again, the paper is usually marked with one or more sample receiving regions. Where a measured volume of blood is applied to the blood collection paper, the marked sample receiving region usually serves as a target. For those cases where the volume of blood is not measured, the marked sample receiving region usually serves as both a target for the user and also to indicate when a sufficient amount of blood has been added to the blood collection paper.

A suboptimal accuracy has been a problem with using current products that employ blood collection paper. Current products often give blood analyte concentrations that differ by as much as 20% or more from the true blood analyte concentration (as usually determined by promptly working up a specimen obtained using venipuncture or arterial puncture in an evacuated blood tube). Although this difference might be acceptable in some cases for medical screening purposes, it is not generally acceptable for medical decision-making purposes. Clearly, if the difference in blood analyte concentration could be lowered, a variety of inexpensive medical diagnostic products based on blood collection paper could be envisioned.

### SUMMARY OF THE INVENTION

Body fluids, such as whole blood, are collected for the determination of analytes contained therein, utilizing a composite collection device which comprises a liquid absorbent web or the like sheet form material and a liquid distributing overlayer contiguous therewith and to which a body fluid sample is applied. Optionally, a stabilizer composition can be distributed in designated sample regions of the web.

The liquid distributing overlayer can be a liquid permeable mesh, coating, film, metal screen, and the like, in contact with the absorbent web and defining a body fluid contact surface. An aliquot of body fluid to be analyzed deposited on the liquid distributing overlayer permeates the overlayer and is absorbed over a relatively large region of the absorbent web. Liquid flux through the overlayer preferably is no more than about 4 microliters/square millimeter/second.

The overlayer is liquid permeable and can be a polymeric coating made of a substantially biologically inert polymer that is substantially non-swellable or insoluble in the body fluid to be collected. Alternatively, the overlayer can be a liquid permeable, porous or perforated polymeric film made from a polymer, such as a polyester, e.g., polyethylene terephthalate, a polyolefin, e.g., polyethylene, polyvinylidene chloride, polycarbonate, polyamide, polyvinyl chloride, polysulfone, polyethersulfone, polystyrene, and the like polymer as well as a frit, sieve, metal screen, and the like.

The absorbent web can be filter paper, a commercially available blood collection paper with or without preservatives and/or binders, and the like.

### BRIEF DESCRIPTION OF THE DRAWINGS

In the drawings,

FIGS. 1-4, inclusive, are enlarged sectional elevations of blood collection paper that illustrate concentration profiles for a single drop blood sample applied to the blood collection paper;

FIG. 5 is an enlarged sectional elevation of blood collection paper that illustrates a concentration profile when several drops of a blood sample are applied to the blood collection paper;

FIG. 6 is a sectional elevation of blood collection paper showing four separate coupons that can be taken from the concentration profile of FIG. 1, and demonstrating variation in quantity of blood in the coupon depending on the location from which the coupon was taken;

FIG. 7 illustrates a blood sample collection device that embodies the present invention;

FIG. 8 illustrates another embodiment of the present invention; and

FIG. 9 illustrates a further embodiment of the present invention in which a blood sample collection card made of a fibrous absorbent web is provided with an overlayer which is perforated polyethylene terephthalate film.

### DESCRIPTION OF PREFERRED EMBODIMENTS

The present improved blood sample collection devices utilize blood collection paper or the like absorbent web for the determination of analytes in bodily fluids such as whole blood, but without being limited thereto. The composite body fluid sample collection device of this invention can be employed for the determination of analytes in other biological fluids as well, such as blood plasma, blood serum, urine, saliva, and amniotic, synovial, spinal, pleural, pericardial, and ascitic fluids.

Current products that employ blood collection paper often give blood analyte concentrations that differ by as much as 20% or more from the true blood analyte concentration. Without being bound by any one theory, it is believed that the reason for this difference is related to noncompliance and also to the varied and irregularly shaped concentration profiles that occur when blood is deposited on the blood collection paper.

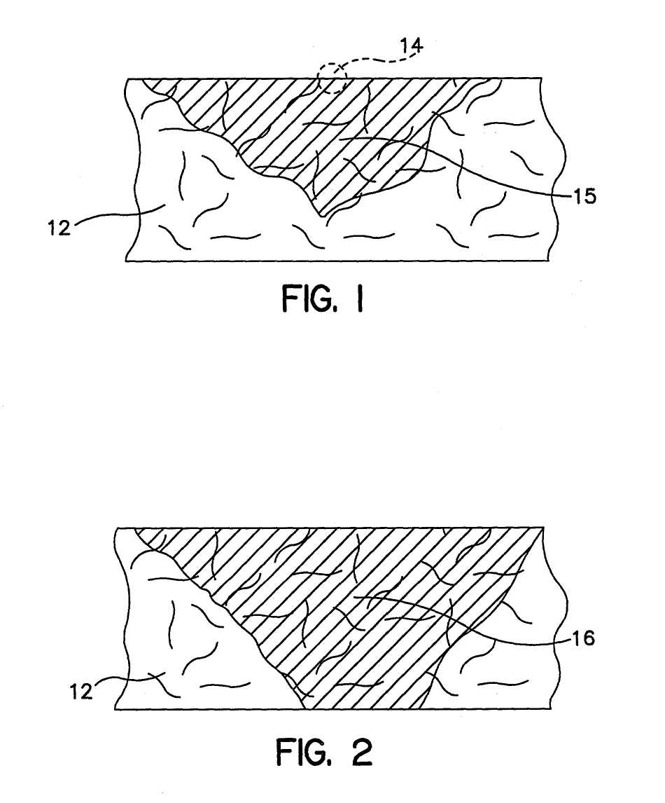

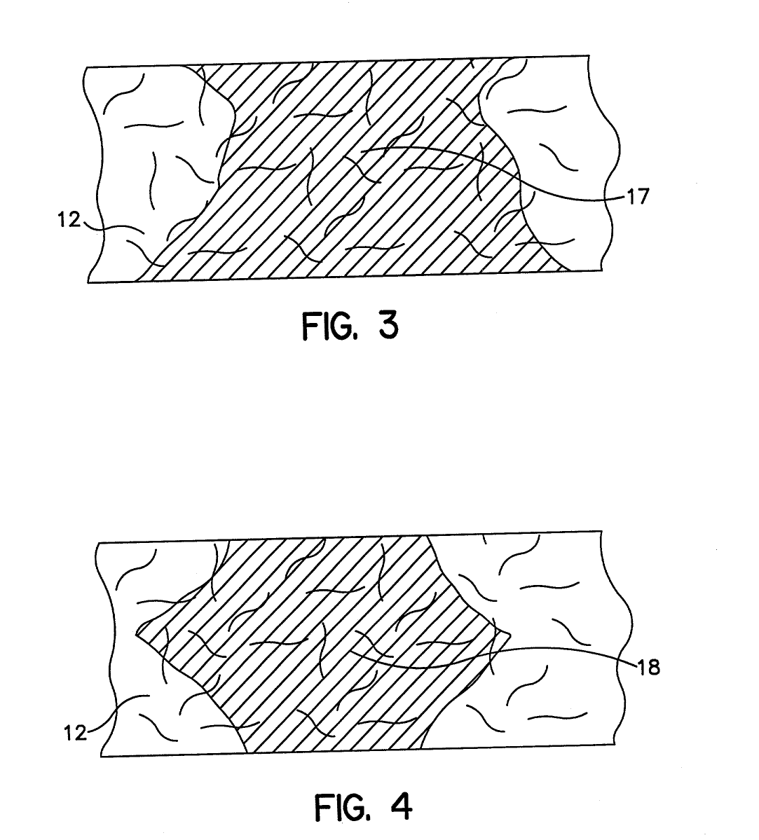

FIGS. 1-5 illustrate some of the ways in which blood concentration profiles can vary in blood collection paper. When one drop of blood is applied to the blood collection paper 12 from an initial contact point 14 and the volume of the drop is insufficient to penetrate the reverse side of the blood collection paper, an irregular cone shaped concentration profile 15, shown in FIG. 1, can be obtained. When the volume of the drop of blood is sufficiently large to penetrate the reverse side of the paper then, depending on the volume of blood in the drop, concentration profiles 16, 17 and 18, shown in FIGS. 2, 3, and 4, respectively, can be obtained.

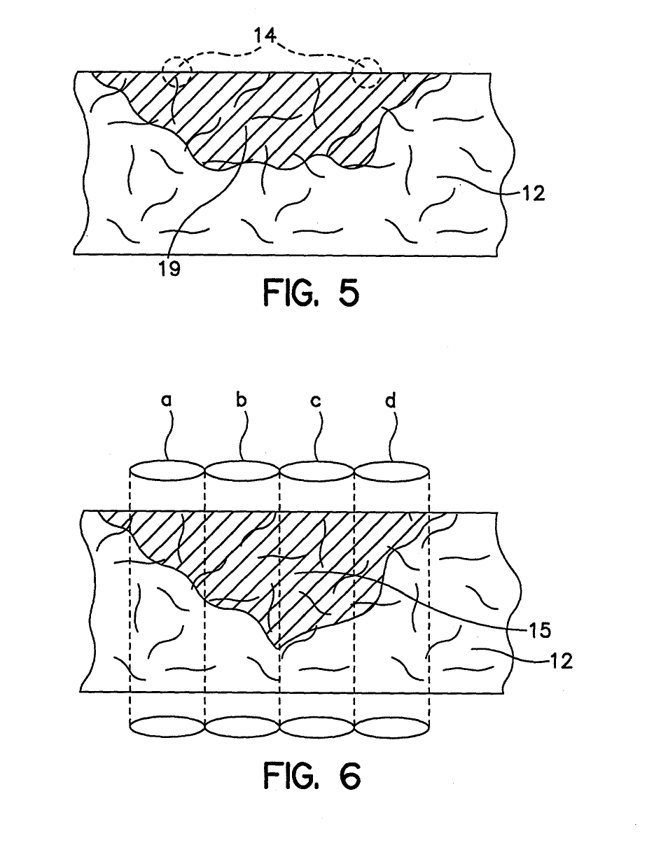

When multiple drops of blood are applied, the concentration profile of the blood in the blood collection paper 12 can be even more irregularly shaped, like the concentration profile 19 illustrated from at least two contact points 14 in FIG. 5, because each drop may have a different volume or because the drops may be wholly or partly overlapping.

Contributing to the occurrence of irregularly shaped concentration profiles are any clotting, drying, wicking, chromatographic, elution, or separation phenomena that occur when blood is applied to the paper, particularly when multiple drops of blood are applied or when the blood over-saturates the paper.

One undesirable phenomenon known as wick-back occurs when the paper becomes over-saturated and the excess blood leaks through the paper and onto the countertop or other surface upon which the paper rests. When the paper is moved, the fluid transferred to the countertop or other surface becomes reabsorbed into the paper unbeknownst to the user.

Typically, a coupon is punched from the blood-containing region of the collection paper, and the analyte of interest is extracted from the coupon and quantitatively determined. Therefore, the presence of an irregularly shaped concentration profile can cause significant error in the determination of the analyte because there is no convenient or inexpensive means of determining whether the coupon that is removed from the blood collection paper for analysis represents the true volume of blood applied to the paper. This is illustrated in FIG. 6 for a cone shaped concentration profile 15 (as in FIG. 1) where coupons (a) through (d) cut from different portions of the blood spotted region of the collection paper 12 would each give different analyte measurement results because the actual volume of blood (or blood residue if dried) in each coupon is different.

The present composite body fluid sample collection device comprises a liquid absorbent web or like sheet form material and a liquid distributing overlayer contiguous therewith. It was found that the inventive composite body fluid sample collection device significantly reduces the difference between the concentration of a blood analyte determined from a blood sample collected in blood collection paper and its true blood analyte concentration. It is believed that this discovery will similarly reduce differences for analytes present in other biological fluid samples collected in blood collection paper. Preferably the liquid absorbent web includes designated sample collection regions.

A preferred composite body fluid sample collection device of this invention employs a liquid permeable, substantially continuous film or coating as a liquid distributing overlayer (overlayer) that permits the biological fluid sample to make multiple contact points with the liquid absorbent web. This way, for a single application of body fluid and also for multiple applications of body fluid, coupons or sections taken for analysis can better represent the actual volume of fluid that was applied. The liquid distributing overlayer causes the body fluid that becomes absorbed into the liquid absorbent web to be more uniformly distributed both across and through the absorbent web over a relative large portion of the sample collection region thereof.

The liquid distributing overlayer defines a body fluid contact surface and can be a liquid permeable mesh, coating or film in contact with the liquid absorbent web. When the overlayer is a film, the film can be hydrophobic or hydrophilic, and can contact the absorbent web as a distinct overlayer, or the film and absorbent web, e.g., paper, may partly interpenetrate one another as an intermediate layer or zone. When the overlayer is a coating, the coating can be a distinct overlayer, or the coating and absorbent web may partly interpenetrate one another. Preferably, the overlayer is a substantially biologically inert, polymeric film that is substantially non-swellable or insoluble in the body fluid to be collected. More preferably, the overlayer is a liquid permeable, porous or perforated polymeric film made from a polymer such as a polyester, e.g., polyethylene terephthalate, a polyolefin, e.g., polyethylene, polyvinylidene chloride, polycarbonate, polyamide, polyvinyl chloride, polysulfone, polyethersulfone, polystyrene, and the like. Preferably, the overlayer is a polyethylene terephthalate film, a polyethylene film or a polyvinylidene chloride

Alternatively the liquid distributing overlayer can be a mesh, e.g., a nylon mesh, or a metal screen, e.g., a copper screen that additionally provides antimicrobial action, a frit, and the like.

Thus, the aliquot of biological fluid to be analyzed is applied to and deposited on the overlayer rather than directly on the liquid absorbent web. The biological fluid passes through apertures or pores in the overlayer, and does not substantially dissolve, dissolve into, swell or chemically interact with the overlayer material. Each such aperture or pore preferably has a size that allows all the components of the biological fluid to pass through the overlayer at a desired flux and be absorbed by the liquid absorbent web. For example, when the specimen is whole blood, each aperture or pore can be of a size that is at least sufficiently large to allow the erythrocytes and other large particles to pass, usually about 8 to about 20 microns in diameter. However, when it is desired to collect hemolyzed blood in the absorbent web, an overlayer having relatively smaller pores can be employed as well. Preferably the absorbent web is a blood collection paper, but other sheet form absorbent materials can also be used.

The pores may have any geometry that will allow the body fluid to make multiple contact points with the paper. This normally means that the pores have a well-defined geometry, such as, for example, the straight through geometry of holes or cylinders that can be formed by punching a plastic film with a punch. However, it is conceivable that the pores can have a poorly defined geometry such as for example the tortuous geometry obtained when a cast film of a dissolved polymer is placed in a non-solvent.

A great advantage of the use of an overlayer is the ability to adjust the resistance of the overlayer so that it may better accommodate certain specific methods of body fluid application or so that it may accommodate a wide variety of body fluid application methods. Designing products that can accommodate a wide variety of application methods is highly desirable for capillary blood specimen collection because it reduces the likelihood of noncompliance errors.

For example, when it is desired that the body fluid be smeared or wiped (or when it is expected that the user would smear or wipe the fluid) then the resistance of the overlayer toward fluid penetration can be made progressively lower from the region where the lens, line, or drop of body fluid initially contacts the overlayer to the region where the body fluid is wiped. This way, a more uniform distribution of the body fluid is obtained across the absorbent web.

Or, for example, when it is desired that the body fluid be dropped (or when it is expected that the user would drop the body fluid) then the resistance of the overlayer toward fluid penetration can be made relatively low which avoids splashing.

As another example, when it is desired that a formed drop of the body fluid be contacted with the overlayer (or when it is expected that the user would contact a formed drop with the overlayer), the resistance of the overlayer toward body fluid penetration can be made relatively high so that the formed drop spreads across the whole overlayer area before the body fluid breaks through to the absorbent web. Preferably, the liquid distributing overlayer has a liquid flux of no more than about 4 microliters/square millimeter/second.

Particularly preferred liquid flux through the overlayer is in the range of about 0.001 and 4 microliters/mm²/second. For whole blood without an anticoagulant, preferred flux is in the range of about 0.004 to about 1 microliter/mm²/second, more preferably in the range of about 0.007 and 0.5 microliters/mm²/second. This way, a more uniform distribution of the body fluid can be obtained across the absorbent web.

In general the resistance of the overlayer toward the penetration of the body fluid may depend on a number of factors or properties that can be selected or adjusted. Among these factors or properties are pore size, pore geometry, the number of pores, the size distribution of pores, the location distribution of the pores, overlayer thickness, overlayer roughness, overlayer interfacial tension with the body fluid, overlayer interfacial tension with air, and the degree of interpenetration of the overlayer and the absorbent web.

Therefore, one example of an overlayer that would be appropriate for the smear mentioned above can consist of a uniform distribution of same-diameter cylindrical pores where the overlayer changes in thickness. Another example can be an overlayer of constant thickness but where the pore diameter varies.

A preferred body fluid collection device embodiment of this invention is a blood collection device that can accommodate a wide variety of whole blood application methods (such as dropping, smearing, wiping, and contacting), A particularly preferred blood collection device comprises S&S® 903™ blood collection paper having a thickness of about 0.45 mm and marked with three collection regions and an overlayer of liquid permeable polyethylene terephthalate film (TELFA CLEAR™ Non-Adherent Wound Dressing, The Kendall Co., Mansfield, Mass.) having a thickness of about 0.03 mm with substantially uniformly distributed pinholes, each pinhole measuring about 0.4 mm in diameter, and a pinhole density of about 0.6 pinholes per square millimeter. The overlayer and blood collection paper are attached, as with a line of glue, in a section that does not interfere with the collection process.

In one study, cholesterol analyses were conducted with the venous and capillary blood specimens collected from eighteen human subjects. The venous specimens were collected in evacuated tubes and analyzed according to standard methods. The capillary specimens were collected in blood collection papers with an overlayer employing the foregoing blood collection device, and for comparison, in blood collection papers without an overlayer, and the specimens were allowed to dry before analysis. The results are given in Table 1.

As shown in Table 1, employing an overlayer gave smaller percent differences between venous and capillary blood analyte concentrations for thirteen of the eighteen subjects. Furthermore, when employing the overlayer, the results from only three subjects exceeded a ten percent difference and no subject had results exceeding a fifteen percent difference. In contrast, without employing the overlayer, the results from eleven subjects exceeded a ten percent difference and the results from six subjects exceeded a fifteen percent difference. Finally, the average percent difference when employing the overlayer was lower than without the overlayer (6.4% with vs 12.9% without).

TABLE 1
Comparison of Cholesterol Analyses

| | Venous | | | | |
|---|---|---|---|---|---|
| | Specimen | Without Overlayer | | With Overlayer | |
| Subject | mg/dL | mg/dL | % Difference | mg/dL | % Difference |
| 1 | 196 | 165 | 15.8 | 207 | −5.6 |
| 2 | 187 | 156 | 16.6 | 180 | 3.7 |
| 3 | 166 | 117 | 29.5 | 153 | 7.8 |
| 4 | 140 | 108 | 22.9 | 133 | 5.0 |
| 5 | 183 | 174 | 4.9 | 210 | −14.8 |
| 6 | 193 | 169 | 12.4 | 193 | 0.0 |
| 7 | 179 | 137 | 23.5 | 167 | 6.7 |
| 8 | 221 | 209 | 5.4 | 237 | −7.2 |
| 9 | 146 | 132 | 9.6 | 163 | −11.6 |
| 10 | 212 | 204 | 3.8 | 240 | −13.2 |
| 11 | 241 | 212 | 12.0 | 220 | 8.7 |
| 12 | 169 | 160 | 5.3 | 177 | −4.7 |
| 13 | 172 | 151 | 12.2 | 187 | −8.7 |
| 14 | 202 | 187 | 7.4 | 220 | −8.9 |
| 15 | 152 | 132 | 13.2 | 150 | 1.3 |
| 16 | 203 | 181 | 10.8 | 210 | −3.4 |
| 17 | 249 | 199 | 20.1 | 247 | 0.8 |
| 18 | 225 | 211 | 6.2 | 230 | −2.2 |

It was also found in the above study that the subjects preferred the process of applying formed drops to the TELFA™ overlayer over the process of applying formed drops directly to the blood collection paper, because the bloody skin did not stick to the overlayer like it did with the paper in the absence of the overlayer. It was further found in the above study that the TELFA™ overlayer could be easily removed from the blood collection paper by the analyst with no loss of any dried blood from the blood collection paper.

In the composite body fluid collection device of this invention, the absorbent web may be provided with one overlayer or more than one, where the overlayers do not overlap and each overlayer independently contacts the absorbent web. When defined target sample receiving regions are desired, such as for blood collection, the blood collection paper can be marked and a transparent overlayer employed. Alternatively, the blood collection paper can be unmarked, and one of either the paper or overlayer can be made to optically contrast such as by making the paper and overlayer different colors or shades. This way, the contrast between the blood collection paper and overlayer can define a target sample receiving region.

When more than one overlayer is present, a different biological fluid can be added to each overlayer. Such a multi-specimen composite collection device is advantageous because it decreases data entry time and also decreases the likelihood of mixups in the laboratory that might occur when separate body fluid collection devices are employed for each body fluid type.

An overlayer, or, each overlayer, when more than one is present, can be in the form of a slip that is brought into contact with the liquid absorbent web (either before or after applying the biological fluid to the overlayer) or can be a film or coating that permanently or semi-permanently contacts the liquid absorbent web.

One advantage of employing an overlayer in the form of a slip is that the slip can be made reusable. Another advantage of applying the body fluid to a slip before the slip is brought into contact with the liquid absorbent web, such as a body fluid collection paper, is that the absorbent web can be spared if an application mistake is made. The slip of overlayer that is brought into contact with the liquid absorbent web may be a self-supporting slip or it may be supported by any convenient means, such as by employing a frame or housing, that does not substantially interfere with the process of contacting the overlayer with the liquid absorbent web, the application of sufficient biological fluid to the overlayer, and with the absorption of body fluid into the absorbent web.

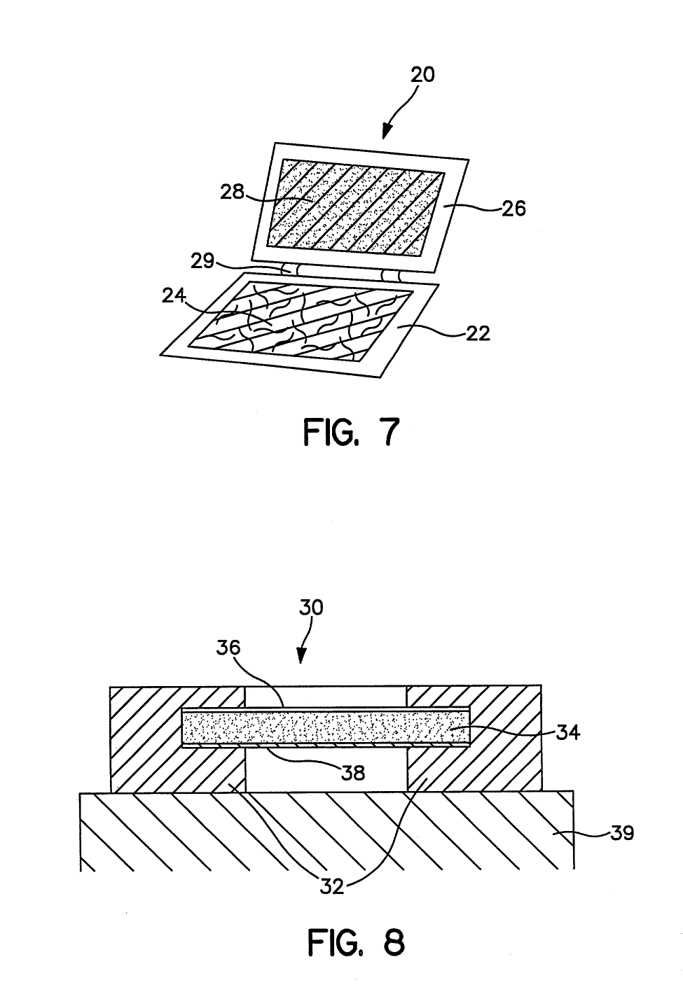

A slip-support can be made to have additional functions that facilitate or enhance the usability or features of products that employ blood collection paper. For example, the slip-support may be made to deliver an exact volume of biological fluid to the overlayer. Also, the slip-support can be made to act as a manifold that distributes the body fluid from a central location to several overlayers. Further, the slip-support can also act as a support for the absorbent web, such as illustrated by the clamshell-like device 20 shown in FIG. 7 comprising a frame-supported body fluid collection paper and frame-supported overlayer in hinged relationship to one another.

Device 20 includes a support frame 22, which can comprise a plastic sheet, a pulp-based cardstock, a cardstock laminated or coated with a polymeric film, or like material. Absorbent web 24 is adhesively secured to support frame 22. Support frame 22 can be a continuous sheet of material over which absorbent web 24 is secured, or support frame 22 can define a substantially rectangular or square-shaped opening, which opening is substantially filled by absorbent web 24.

A slip frame 26 surrounds overlayer 28, which is secured thereto. Slip frame 26 is moveably attached to support frame 22 by one or more hinges 29. Slip frame 26, containing overlayer 28 can be positioned so as to contact overlayer 28 with absorbent web 24, or can be positioned with overlayer 28 and absorbent web 24 separated as shown in FIG. 7.

While employing an overlayer in the form of a slip is often highly advantageous, in some cases, particularly where the users are highly inexperienced, there is a risk that the user may misapply the body fluid directly to the absorbent web. Therefore, in order to prevent such misapplication, it is preferable to employ an overlayer that is a film or coating that semi-permanently or permanently contacts the liquid absorbent web.

When the overlayer is a film or coating that is semi-permanently or permanently contiguous with the absorbent web, the overlayer-absorbent web composite can be self-supporting. Alternatively, the overlayer-absorbent web composite can be supported by any convenient means, such as by the use of a frame or housing, as illustrated by device 30 in FIG. 8.

Device 30 comprises a frame 32 containing absorbent web 34. Overlayer 36 is secured to the upper surface of absorbent web 34 by frame 32. Backing layer 38, is secured to the lower surface of absorbent web 34 by frame 32. Alternatively, backing layer 38 can be adhesively secured to absorbent web 34.

The overlayer-absorbent web composite-support can be made to have additional functions that facilitate or enhance the usability of the device or features of products that employ blood collection paper. For example, like the slip-support described above, the overlayer-absorbent web composite-support can be made to deliver an exact volume of biological fluid or act as a manifold. Further, the overlayer-absorbent web composite support can provide or facilitate contact between the overlayer and absorbent web. Also, the overlayer-absorbent web composite support can serve to elevate the backside of the body fluid collection device 30 from the working countertop 39 as illustrated in FIG. 8 thereby facilitating drying and eliminating wick-back.

The absorbent web 34 of the composite can also be permanently or semi-permanently backed with a film or coating to provide a backing layer 38 (FIG. 8) that minimizes or prevents the unintended loss of the fluid through the back of the web at least during the collection process, but not necessarily during a drying process if one is employed. The backing layer 38 can be utilized with or without a frame 32, as desired. This is advantageous because the loss of the sampled fluid during the sampling process can significantly contribute to inaccurate analyte quantification. This is also advantageous because the presence of the backing layer 38 can minimize noncompliance errors because the device 30 can be placed on virtually any work surface, including absorbent surfaces, without the loss of fluid. This is further advantageous because it helps minimize or prevent contamination, both of the composite by substances present on the work surface and of the work surface by substances leaked from the composite. While the backing layer 38 is non-permeable to the biological fluid and its components, it may be made porous to gas in order to facilitate drying when drying of the specimen is desired. Further, the backing layer 38 can be transparent, opaque, or translucent. The backing layer 38 is preferably transparent in capillary blood collection situations so that the user and the lab can verify whether the blood sample has penetrated the web 34.

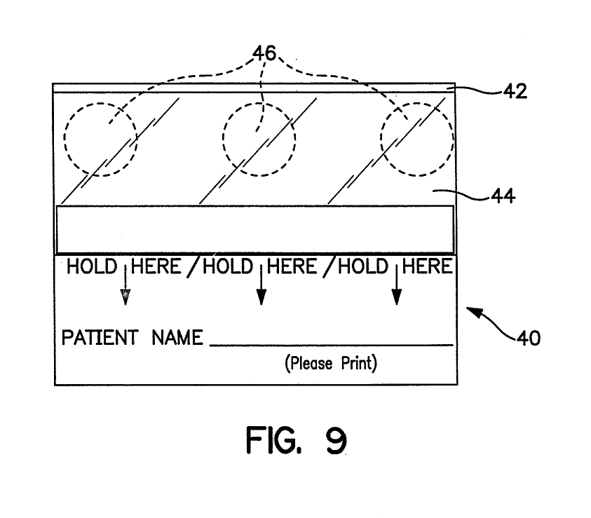

A preferred embodiment of a composite body fluid collection device is illustrated in FIG. 9 made in the form of a capillary blood sample collection card 40 comprised of a fibrous absorbent web 42 provided with a perforated overlayer 44. Preferably, the overlayer 44 is perforated polyethylene terephthalate film and the absorbent web 42 is a blood collection paper, such as S&S® 903™ paper. Indicia can be imprinted on the card providing the user with instructions for use and patient identification. Preferably, the card 40 comprises designated sample collection regions 46 imprinted on either the web 42, the overlayer 44 or both.

Preferably the composite body fluid collection device is provided in a kit, together with an instruction manual, and appropriate accessories, such as lancets, lancet disposal container, alcohol prep wipe, sterile gauge and adhesive bandage, containers for delivery of sample to the analytical laboratory, and the like, for the user.

We claim:

1. An article of manufacture for collection of body fluid samples which comprises
a liquid absorbent web; and
a liquid distributing overlayer contiguous with the absorbent web;
the liquid distributing overlayer being liquid permeable and providing a liquid flux through the overlayer of no more than about 4 microliters/mm²/second.

2. The article of manufacture in accordance with claim 1 wherein the liquid distributing overlayer is a polymeric coating.

3. The article of manufacture in accordance with claim 2 wherein the polymeric coating is constituted by a polymer that is substantially non-swellable in the body fluid.

4. The article of manufacture in accordance with claim 2 wherein the polymeric coating is constituted by a polymer that is substantially insoluble in the body fluid.

5. The article of manufacture in accordance with claim 1 wherein the liquid distributing overlayer is a film permeable by the body fluid.

6. The article of manufacture in accordance with claim 5 wherein the film is a polyethylene terephthalate film.

7. The article of manufacture in accordance with claim 5 wherein the film is a polyethylene film.

8. The article of manufacture in accordance with claim 5 wherein the film is a polyvinylidene chloride film.

9. The article of manufacture in accordance with claim 1 wherein the liquid distributing overlayer is a mesh.

10. The article of manufacture in accordance with claim 1 wherein the liquid distributing overlayer is a metal screen.

11. The article of manufacture in accordance with claim 1, wherein the liquid absorbent web is blood collection paper.

12. An article of manufacture for collection of body fluid samples which comprises
a liquid absorbent web having designated sample regions; and
a liquid distributing overlayer contiguous with the absorbent web;
the liquid distributing overlayer being liquid permeable and providing a liquid flux through the overlayer of no more than about 4 microliters/mm²/second.

13. An article of manufacture in accordance with claim 12 wherein the liquid distributing overlayer is a polymeric coating.

14. An article of manufacture in accordance with claim 12 wherein the liquid distributing overlayer is a film that is permeable by the body fluid.

15. An article of manufacture in accordance with claim 14 wherein the film is selected from the group consisting of a polyethylene terephthalate film, a polyethylene film, and a polyvinylidene chloride film.

16. An article of manufacture in accordance with claim 12 wherein the liquid distributing overlayer is a mesh or a metal screen.

17. An article of manufacture in accordance with claim 12 wherein the liquid absorbent web is a blood collection paper.

18. An article of manufacture in accordance with claim 12 further comprising a stabilizing composition distributed in the designated sample collection regions of the liquid absorbent web.

19. An article of manufacture for collection of a body fluid sample which comprises
a support frame having an upper surface;
a liquid absorbent web secured to the upper surface of the support frame;
a liquid distributing overlayer;
a slip frame surrounding the overlayer and secured thereto; and
the support frame being in a hinged relationship to the slip frame such that the overlayer can be brought into contact with the absorbent web; and
the liquid distributing overlayer being liquid permeable and providing a liquid flux through the overlayer of no more than about 4 microliters/mm²/second.

20. An article of manufacture in accordance with claim 19 wherein the overlayer is a polymeric film permeable by a body fluid.

21. An article of manufacture in accordance with claim 20 wherein the polymeric film is selected from the group consisting of a polyethylene terephthalate film, a polyethylene film, and a polyvinylidene chloride film.

22. An article of manufacture in accordance with claim 19 wherein the overlayer is a liquid permeable polyethylene terephthalate film having substantially uniformly distributed pinholes, each pinhole measuring about 0.4 mm in diameter, and having a pinhole density of about 0.6 pinholes per square millimeter.

23. An article of manufacture in accordance with claim 19 wherein the absorbent web is a blood collection paper.

24. An article of manufacture for collection of body fluid samples which comprises:
a liquid absorbent web;
a liquid distributing overlayer contiguous with the absorbent web; and
a backing layer contiguous with the absorbent web on the side of the web opposite the liquid distributing overlayer;
the web, overlayer, and backing layer being supported by a frame; and
the liquid distributing overlayer being liquid permeable and providing a liquid flux through the overlayer of no more than about 4 microliters/mm²/second.

25. An article of manufacture in accordance with claim 24 wherein the overlayer is a polymeric film permeable by a body fluid.

26. An article of manufacture in accordance with claim 25 wherein the film is selected from the group consisting of a polyethylene terephthalate film, a polyethylene film, and a polyvinylidene chloride film.

27. An article of manufacture in accordance with claim 24 wherein the overlayer is a polyethylene terephthalate film having substantially uniformly distributed pinholes measuring about 0.4 mm in diameter, and having a pinhole density of about 0.6 pinholes per square millimeter.

28. An article of manufacture in accordance with claim 24 wherein the absorbent web is a blood collection paper.

29. A kit comprising at least one body fluid collection device, at least one container for shipping the body fluid collection device to a clinical laboratory for analysis of a body fluid analyte, printed instructions for obtaining a body fluid sample and for depositing the body fluid sample in the body fluid collection device and instructions for shipping the body fluid collection device containing a body fluid sample to a clinical laboratory for analysis; the body fluid collection device comprising:
a liquid absorbent web; and
a liquid distributing overlayer contiguous with the absorbent web;
the liquid distributing overlayer being liquid permeable and providing a liquid flux through the overlayer of no more than about 4 microliters/mm²/second.

30. A kit in accordance with claim 29 wherein the liquid absorbent web of the body fluid collection device has a designated body fluid sample collection region having a stabilizing composition distributed therein.
